SEQUENCE LISTING (2 sequences)

SEQ ID NO: 1 - protein, 301 aa, Synthetic construct
MHHHHHHRIAFIGLGNMGAPMARNLIKAGHQLNLFDLNQTVLAELAELGGQVSASPKDAAASSELVITMLPAAAHVRSVYLGDDGVLAGVRPGTPTVDCSTIDPQTAREVSKAAAAKGVDMGDAPVSGGTGGAAAGTLTFMVGASAELFAALKPVLEQMGRNIVHCGEVGTGQIAKICNNLLLGISMIGVSEAMALGNALGIDTQVLAGIINSSTGRCWSSDTYNPWPGIIETAPASRGYTGGFGAELMLKDLGLATEAARQAHQPVIMGALAQQLYQAMSLRGDGGKDFSAIVEGYRKKD

SEQ ID NO: 2 - DNA, 906 bp, Synthetic construct
  source 1..906: mol_type = other DNA
atgcatcatcaccaccaccacaggatagctttcatcggcttaggtaatatgggcgctccgatggcgcgcaacctgattaaagcaggccaccaactgaatctgttcgatctgaaccagaccgttctggccgagttggcggagttgggcggccaagtttctgcctccccgaaagacgccgctgcgagcagcgagttggtgattaccatgctgccggcggctgcgcatgttagaagcgtttatctgggcgatgatggtgttctcgcgggcgtacgccctggtactccgacggtggattgcagcaccattgatccgcaaaccgcacgtgaagtgtccaaagcggcggctgctaaaggtgttgatatgggcgacgcaccggtgtctggtggaacgggtggcgccgcggctggtactctgacctttatggttggtgcgagcgctgagttgtttgctgcgttgaagccggtgctggaacagatgggtcgtaatatcgtgcactgcggtgaagtgggcacgggtcaaattgcaaagatctgcaataacctgctgttgggtatcagcatgattggtgtctcggaggccatggcgctgggtaacgcattggggatcgacacccaggtcctggcgggcattatcaacagctccaccggtcgttgttggtcatccgacacctataacccgtggccggggatcatcgaaaccgcgccagcatctcgtggttacaccggtggttttggtgcggagctgatgctgaaggacctgggtctggctaccgaagctgcgcgtcaggcgcatcaaccggtcatcatgggcgcgctcgcgcagcagttataccaggccatgagcctgcgtggcgacggcggcaaggacttcagcgcgattgttgaaggttaccgcaaaaaggactaa

(19) United States
(12) Patent Application Publication
(10) Pub. No.: US 2025/0059574 A1
(43) Pub. Date: Feb. 20, 2025

(54) METHODS FOR PREPARING (2S,3R)-P-METHYLSULFONYLPHENYLSERINE

(71) Applicants: TAIZHOU UNIVERSITY, Taizhou (CN); Taizhou Lingfeng Biotechnology Co., Ltd., Taizhou (CN)

(72) Inventors: Zhongyi YANG, Taizhou (CN); Shaoyang ZHANG, Taizhou (CN); Yi WEI, Taizhou (CN); Kun CHENG, Taizhou (CN); Jieyang LU, Taizhou (CN); Ting YANG, Taizhou (CN); Weijin HUANG, Taizhou (CN); Qingfeng CAI, Taizhou (CN)

(73) Assignees: TAIZHOU UNIVERSITY, Taizhou (CN); Taizhou Lingfeng Biotechnology Co., Ltd., Taizhou (CN)

(21) Appl. No.: 18/795,140

(22) Filed: Aug. 5, 2024

(30) Foreign Application Priority Data
Aug. 16, 2023 (CN) 202311031949.3

(51) Int. Cl. C12P 13/06

(52) U.S. Cl. CPC C12P 13/06

(57) ABSTRACT

A method for preparing (2S,3R)-p-methylsulfonylphenylserine is provided. In order to solve the existing problem of low conversion rate, provided is a method for preparing (2S,3R)-p-methylsulfonylphenylserine, including carrying out a reaction between p-methylsulfonylbenzaldehyde, L-threonine and pyridoxal phosphate in an oxygen-containing environment under the combined action of transaldolase and acetaldehyde oxidase to obtain a product (2S,3R)-p-methylsulfonylphenylserine. The method can effectively achieve the effect of improving the conversion rate of the reaction and has the advantage of high product yield.

### CROSS REFERENCE TO THE RELATED APPLICATIONS

This application is based upon and claims priority to Chinese Patent Application No. 202311031949.3, filed on Aug. 16, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named YFZT0201_Sequence_Listing_20240809.xml, created on Aug. 9, 2024, and is 4,053 bytes in size.

### TECHNICAL FIELD

The present disclosure relates to a method for preparing (2S,3R)-p-methylsulfonylphenylserine, and belongs to the technical field of pharmaceutical synthesis.

### BACKGROUND

(2S,3R)-p-methylsulfonylphenylserine (L-p-methylsulfonylphenylserine) is a key intermediate in the synthesis of a veterinary drug florfenicol and thiamphenicol, and can be obtained by resolution of D,L-p-methylsulfonylphenylserine, or by asymmetric synthesis. Among asymmetric synthesis methods, bioconversion is currently the most efficient method, and main enzymes used include aldolase, transaldolase, and the like. A reaction catalyzed by the transaldolase takes p-methylsulfonylbenzaldehyde and L-threonine as main raw materials, and due to the introduction of one chiral carbon atom by a substrate molecule, its reaction product has a higher ee value and a higher de value, having better industrial application prospects.

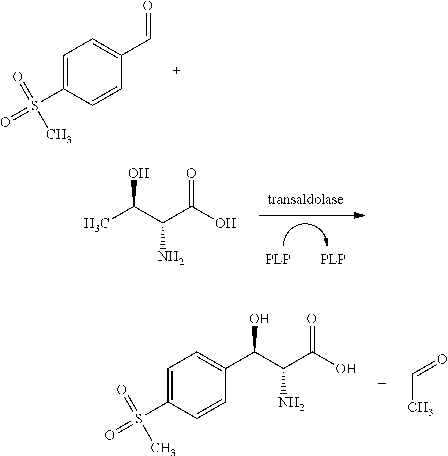

However, acetaldehyde produced by this reaction has a very large inhibitory effect on the activity of the transaldolase used in the reaction system. In order to solve this inhibitory effect, alcohol dehydrogenase is generally used to convert acetaldehyde into relatively less toxic ethanol, but the reaction requires NADH, NAD or the like as a coenzyme, and in order to reduce the amount of the coenzyme used and save cost, a regeneration system of coenzymes such as glucose dehydrogenase and formate dehydrogenase is generally introduced.

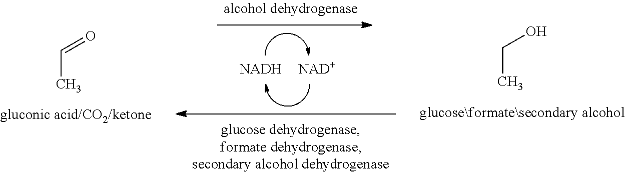

As can be seen from the above description, although the existing transaldolase route is the first choice for industrialization, there are still multiple enzymes and multiple coenzymes such as NAD and NADH that need to be used, which increases the complexity of the reaction and the production, especially the need for the expensive coenzyme NAD, greatly increases the cost of the process, and severely affects the competitiveness and conversion efficiency of the biocatalytic process.

### SUMMARY

In view of the defects existing in the prior art, the present disclosure provides a method for preparing (2S,3R)-p-methylsulfonylphenylserine, which solves the problem of how to improve the conversion efficiency without the need to add a coenzyme NAD, which is conducive to reducing the cost.

An object of the present disclosure is achieved by the following technical solution:
provided is a method for preparing (2S,3R)-p-methylsulfonylphenylserine, including the step of:
carrying out a reaction between p-methylsulfonylbenzaldehyde, L-threonine and pyridoxal phosphate in an oxygen-containing environment under the combined action of transaldolase and acetaldehyde oxidase to obtain a product (2S,3R)-p-methylsulfonylphenylserine.

The method of the present disclosure enables efficient reaction of p-methylsulfonylbenzaldehyde and L-threonine to form the corresponding product (2S,3R)-p-methylsulfonylphenylserine by employing an enzyme system of transaldolase and acetaldehyde oxidase, with high biocatalytic conversion; meanwhile, although the presence of acetaldehyde, which is a byproduct produced during the reaction, has an inhibitory effect on the reaction, the byproduct acetaldehyde can be efficiently converted into acetic acid in an environment where oxygen exists by the acetaldehyde oxidase added, and by adding the aforementioned acetaldehyde oxidase, the reaction process does not require the addition of the coenzyme NAD and a regeneration system of coenzymes, not only can the reaction be carried out efficiently, but also the byproduct acetaldehyde can be effectively removed, and the conversion rate of the reaction can be more efficiently improved, and thus, the method has the effects of high product yield and high atom utilization rate, and the reaction system of the present disclosure does not require the addition of the coenzyme NAD and the regeneration system, reducing the production cost and simplifying the operation, and also reducing the types of three wastes (wastewaters, exhaust gases, and solid wastes) generated, which is more conducive to industrial production. The above transaldolase may be transaldolase commonly used in the synthesis of (2S,3R)-p-methylsulfonylphenylserine, preferably transaldolase (an enzyme number EC.2.2.1.2). More importantly, the addition of the acetaldehyde oxidase in the present disclosure not only enables the reaction to be carried out more efficiently, but also achieves the effects without the needs of the addition of regeneration systems such as the coenzyme NAD, and without the needs of the addition of the regeneration system of coenzymes such as glucose, sodium formate and isopropanol into the reaction system, thereby reducing the production cost more effectively.

In the above method for preparing (2S,3R)-p-methylsulfonylphenylserine, the acetaldehyde oxidase is selected from one or more of acetaldehyde oxidase having an amino acid sequence shown in SEQ ID NO: 1, an azalea plant extract and a Schefflera octophylla plant extract.

By employing the acetaldehyde oxidase having the amino acid sequence, or the plant extract also containing acetaldehyde oxidase, the conversion of acetaldehyde produced during the reaction into acetic acid can also be effectively achieved by directly adding the plant extract described above, so that the reaction can be carried out efficiently without the need of additional addition of the coenzyme NAD and the like, facilitating efficient conversion of the reaction, and also having better product yield and quality.

In the above method for preparing (2S,3R)-p-methylsulfonylphenylserine, the oxygen-containing environment is an environment in which air or oxygen is introduced.

The air contains oxygen. Under the environment where air or oxygen exists, the reaction is carried out in the presence of oxygen, which enables the byproduct acetaldehyde produced to react with the oxygen in the gas introduced to be converted into the corresponding acetic acid, thereby effectively avoiding the phenomenon of inhibition of the reaction by the presence of acetaldehyde, allowing the reaction to be carried out efficiently, and also contributing to better increase the efficiency and conversion rate of the reaction.

In the above method for preparing (2S,3R)-p-methylsulfonylphenylserine, catalase is also added into raw materials of the reaction.

Here, catalase can be used as long as it can allow oxydol (hydrogen peroxide), a byproduct that may be present in the system, to be hydrolyzed into oxygen and water by the reaction. Since a small amount of hydrogen peroxide byproduct will be produced in the process of converting acetaldehyde into acetic acid, in order to better improve the conversion rate of the reaction, catalase is added to the reaction system to decompose the byproduct hydrogen peroxide into water and oxygen, effectively avoiding the presence of hydrogen peroxide, and making the reaction process more conducive to being carried out in the direction of synthesis of (2S,3R)-p-methylsulfonylphenylserine.

Wherein the acetaldehyde oxidase, catalase and transaldolase are used in amounts such that the reaction conversion rate can reach a higher level, for example, the acetaldehyde oxidase, catalase and transaldolase are used in amounts such that the reaction conversion rate reaches 80% or more or 90% or more. Moreover, the amounts of the acetaldehyde oxidase, catalase and transaldolase used need to control the reaction time to be within 24 h. Because of its wide application, the catalase has been supplied in many commercial products, and its price is low, so the catalase is generally used in excess in the reaction.

In the above method for preparing (2S,3R)-p-methylsulfonylphenylserine, the reaction is carried out at a temperature of 30° C.-40° C. The reaction temperature has the advantages of mild reaction conditions and easy operation, which can better exert the catalytic ability of enzymes and have the effect of excellent conversion rate.

In the above method for preparing (2S,3R)-p-methylsulfonylphenylserine, the reaction is carried out in water. It is more advantageous to carry out the reaction in the water, and directly using the water as a solvent system for the reaction is environmentally friendly, which is more advantageous to reduce environmental pollution.

In the above method for preparing (2S,3R)-p-methylsulfonylphenylserine, the reaction is carried out at a pH of 6.5-9.0. Thus, the enzyme catalytic ability of the acetaldehyde oxidase, transaldolase and catalase added can be better maintained, and the effect of high catalytic activity can be achieved.

In the above method for preparing (2S,3R)-p-methylsulfonylphenylserine, a mass ratio of p-methylsulfonylbenzaldehyde to L-threonine to pyridoxal phosphate is 6.0:(4.0-5.0):(0.1-0.3).

A chemical n equation for the above method for preparing (2S,3R)-p-methylsulfonylphenylserine of the present disclosure is shown below:

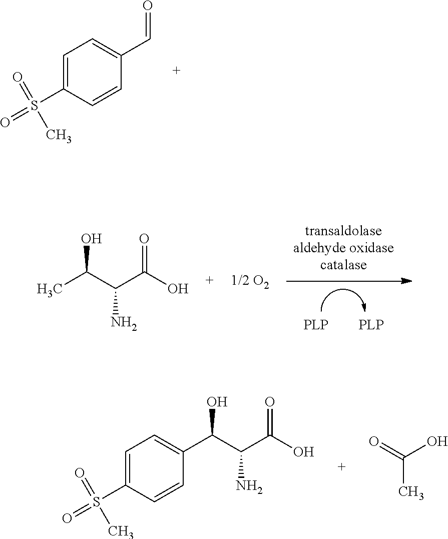

In the above reaction equation, acetaldehyde produced during the reaction of substrates can be efficiently converted into acetic acid under the action of the acetaldehyde oxidase added, and a specific chemical reaction equation is shown below:

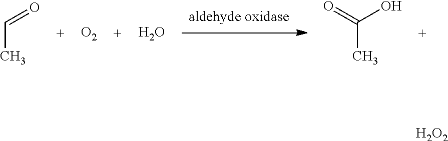

In summary, compared with the prior art, the present disclosure has the following advantages:
1. By using the acetaldehyde oxidase as an enzyme catalytic additive, in conjunction with the added transaldolase, the effect of increasing the conversion rate of the reaction can be effectively achieved, and the product yield is high.
2. By adding the catalase in the reaction system, the byproduct oxydol (hydrogen peroxide) can be better decomposed into water and oxygen, thereby effectively avoiding the presence of the byproduct hydrogen peroxide, which is beneficial to allowing the reaction to be carried out in the direction of synthesis of (2S,3R)-p-methylsulfonylphenylserine.

### DETAILED DESCRIPTION OF THE EMBODIMENTS

The technical solutions of the present disclosure are further specifically illustrated below by specific examples, but the present disclosure is not limited to these examples.

### Example 1

20 mL of a recombinant Escherichia coli strain containing a T7 promoter and expressing acetaldehyde oxidase was inoculated into a TB medium, and then, activated culture was performed for 2 h at a temperature controlled at 37° C. under aeration and stirring, the temperature was reduced to 25° C., IPTG (isopropyl-beta-D-thiogalactopyranoside) with a final concentration of 0.5 mmol/L was added, and fermentation culture was continued to be performed at 25° C. for 20 h to obtain 2.1 L of a fermentation broth after the end of the culture. Thalli were collected by centrifugation and stored at −20° C. for later use.

20 g of the obtained thalli was resuspended in 80 mL of 50 mmol/L potassium phosphate buffer at a pH of 7.0, and then subjected to ultrasonic wall breaking to obtain a corresponding wall-broken enzyme solution, which was then centrifuged at 10000 rpm for 10 min to obtain an enzyme solution for later use.

Preferably, an amino acid sequence of the acetaldehyde oxidase described above is as shown in SEQ ID NO: 1 below:

MHHHHHHRIAFIGLGNMGAPMARNLIKAGHQLNLFDLNQTVLAELAELG
GQVSASPKDAAASSELVITMLPAAAHVRSVYLGDDGVLAGVRPGTPTVD
CSTIDPQTAREVSKAAAAKGVDMGDAPVSGGTGGAAAGTLTFMVGASAE
LFAALKPVLEQMGRNIVHCGEVGTGQIAKICNNLLLGISMIGVSEAMAL
GNALGIDTQVLAGIINSSTGRCWSSDTYNPWPGIIETAPASRGYTGGFG
AELMLKDLGLATEAARQAHQPVIMGALAQQLYQAMSLRGDGGKDFSAIV
EGYRKKD.

A nucleotide sequence of the acetaldehyde oxidase described above is shown in SEQ ID NO: 2 below:

ATGCATCATCACCACCACCACAGGATAGCTTTCATCGGCTTAGGTAATA
TGGGCGCTCCGATGGCGCGCAACCTGATTAAAGCAGGCCACCAACTGAA
TCTGTTCGATCTGAACCAGACCGTTCTGGCCGAGTTGGCGGAGTTGGGC
GGCCAAGTTTCTGCCTCCCCGAAAGACGCCGCTGCGAGCAGCGAGTTGG
TGATTACCATGCTGCCGGCGGCTGCGCATGTTAGAAGCGTTTATCTGGG
CGATGATGGTGTTCTCGCGGGCGTACGCCCTGGTACTCCGACGGTGGAT
TGCAGCACCATTGATCCGCAAACCGCACGTGAAGTGTCCAAAGCGGCGG
CTGCTAAAGGTGTTGATATGGGCGACGCACCGGTGTCTGGTGGAACGGG
TGGCGCCGCGGCTGGTACTCTGACCTTTATGGTTGGTGCGAGCGCTGAG
TTGTTTGCTGCGTTGAAGCCGGTGCTGGAACAGATGGGTCGTAATATCG
TGCACTGCGGTGAAGTGGGCACGGGTCAAATTGCAAAGATCTGCAATAA
CCTGCTGTTGGGTATCAGCATGATTGGTGTCTCGGAGGCCATGGCGCTG
GGTAACGCATTGGGGATCGACACCCAGGTCCTGGCGGGCATTATCAACA
GCTCCACCGGTCGTTGTTGGTCATCCGACACCTATAACCCGTGGCCGGG
GATCATCGAAACCGCGCCAGCATCTCGTGGTTACACCGGTGGTTTTGGT
GCGGAGCTGATGCTGAAGGACCTGGGTCTGGCTACCGAAGCTGCGCGTC
AGGCGCATCAACCGGTCATCATGGGCGCGCTCGCGCAGCAGTTATACCA
GGCCATGAGCCTGCGTGGCGACGGCGGCAAGGACTTCAGCGCGATTGTT
GAAGGTTACCGCAAAAAGGACTAA.

### Example 2

### Regarding the Effect of Acetaldehyde on the Activity of Transaldolase

In a 50 mL round-bottom flask, 10 mL of a reaction system contained 14 g/L p-methylsulfonylbenzaldehyde, 10.5 g/L threonine, 1 g/L pyridoxal phosphate, and 1 mL of transaldolase (Taizhou Lingfeng Biotechnology Co., Ltd., LF145), different concentrations (0, 1 g/L, 5 g/L, 10 g/L, and 20 g/L) of acetaldehyde were respectively prepared, the mixture was shaken in a water bath at 35° C. for 10 min, and the content of D-acid produced was detected by HPLC.

According to the different concentrations of acetaldehyde described above, a reaction was carried out for 10 min without the addition of acetaldehyde to produce 3.31 g/L (2S,3R)-p-methylsulfonylphenylserine;
a reaction was carried out for 10 min after addition of 1 g/L acetaldehyde to produce 1.76 g/L (2S,3R)-p-methylsulfonylphenylserine;
a reaction was carried out for 10 min after addition of 5 g/L acetaldehyde to produce 0.66 g/L (2S,3R)-p-methylsulfonylphenylserine;
a reaction was carried out for 10 min after addition of 10 g/L acetaldehyde to produce 0.303 g/L (2S,3R)-p-methylsulfonylphenylserine; and
a reaction was carried out for 10 min after addition of 20 g/L acetaldehyde to produce 0.153 g/L (2S,3R)-p-methylsulfonylphenylserine.

Thus, it can be seen that the amount of acetaldehyde present in the above reaction system will inhibit the production of the product (2S,3R)-p-methylsulfonylphenylserine. Therefore, removal of acetaldehyde is critical to the reaction.

### Example 3

6 g of p-methylsulfonylbenzaldehyde, 4.5 g of L-threonine, 0.1 g of pyridoxal phosphate, 15 ml of transaldolase (purchased from Taizhou Lingfeng Biotechnology Co., Ltd., LF145), 15 ml of aldehyde oxidase (a supernatant obtained after wall breaking and centrifugation, i.e., the enzyme solution obtained by the method of Example 1), 65 ml of ddH2O and 5 ml of catalase (purchased from Novozymes and having an enzyme activity of 60000 SCIU/mL) were added into a clean reaction flask, with a total volume of 100 ml, a gas (air) was introduced into the reaction system, a pH of the system was adjusted to be 7.0, the reaction system was controlled to be subjected to a reaction at a temperature of 35° C., and the reaction was terminated after 21 hours of the reaction. The resulting reaction solution was detected, and the results showed that the concentration of (2S,3R)-p-methylsulfonylphenylserine was 108.942 g/l, the final volume was 63 ml, and a conversion rate of (2S,3R)-p-methylsulfonylphenylserine was 81.26%.

### Example 4

6 g of p-methylsulfonylbenzaldehyde, 4.5 g of L-threonine, 0.1 g of pyridoxal phosphate, 15 ml of transaldolase (purchased from Taizhou Lingfeng Biotechnology Co., Ltd., LF145), 15 ml of aldehyde oxidase (a supernatant obtained after wall breaking and centrifugation, i.e., the enzyme solution obtained by the method of Example 1), 65 ml of ddH2O and 5 ml of catalase (purchased from Novozymes and having an enzyme activity of 60000 SCIU/mL) were added into a clean reaction flask, with a total volume of 100 ml. A gas (air) was introduced into the reaction system, a pH of the system was adjusted to be 7.5, the reaction system was controlled to be subjected to a reaction at a temperature of 35° C., and the reaction was terminated after 19 hours of the reaction. The resulting reaction solution was detected, and the results showed that the concentration of (2S,3R)-p-methylsulfonylphenylserine was 79.290 g/l, the final volume was 90 ml, and a conversion rate of (2S,3R)-p-methylsulfonylphenylserine was 84.49%.

### Example 5

6 g of p-methylsulfonylbenzaldehyde, 4.5 g of L-threonine, 0.1 g of pyridoxal phosphate, 15 ml of transaldolase (purchased from Taizhou Lingfeng Biotechnology Co., Ltd., LF145), 15 ml of aldehyde oxidase (a supernatant obtained after wall breaking and centrifugation, i.e., the enzyme solution obtained in Example 1), 65 ml of ddH2O and 5 ml of catalase (purchased from Novozymes and having an enzyme activity of 60000 SCIU/mL) were added into a clean reaction flask, with a total volume of 100 ml, a pH of the reaction system was adjusted to be 8.0, a reaction was carried out at a temperature controlled to be 35° C., a gas (air) being introduced during the reaction, and the reaction was finished after 17 hours of the reaction. The resulting reaction solution was detected, and the results showed that the concentration of (2S,3R)-p-methylsulfonylphenylserine was 80.030 g/l, the final volume was 102 ml, and a conversion rate of (2S,3R)-p-methylsulfonylphenylserine was 96.65%.

### Example 6

6 g of p-methylsulfonylbenzaldehyde, 4.5 g of L-threonine, 0.1 g of pyridoxal phosphate, 15 ml of transaldolase (purchased from Taizhou Lingfeng Biotechnology Co., Ltd., LF145), 15 ml of aldehyde oxidase (a supernatant obtained after wall breaking and centrifugation, i.e., the enzyme solution obtained by the method of Example 1), 65 ml of ddH2O and 5 ml of catalase (purchased from Novozymes and having an enzyme activity of 60000 SCIU/mL) were added into a clean reaction flask, with a total volume of 100 ml. A gas (air) was introduced into the reaction system, a pH of the reaction system was adjusted to be 9.0, a reaction was carried out at a temperature controlled to be 35° C., and the reaction was finished after 23.5 hours of the reaction. The resulting reaction solution was detected, and the results showed that the concentration of (2S,3R)-p-methylsulfonylphenylserine was 84.177 g/l, the final volume was 92 ml, and a conversion rate of (2S,3R)-p-methylsulfonylphenylserine was 91.70%.

### Example 7

6 g of p-methylsulfonylbenzaldehyde, 4.5 g of L-threonine, 0.1 g of pyridoxal phosphate, 15 ml of transaldolase (purchased from Taizhou Lingfeng Biotechnology Co., Ltd., LF145), 15 ml of aldehyde oxidase (a supernatant obtained after wall breaking and centrifugation, i.e., the enzyme solution obtained by the method of Example 1), 65 ml of ddH2O and 5 ml of catalase (purchased from Novozymes and having an enzyme activity of 60000 SCIU/mL) were added into a clean reaction flask, with a total volume of 100 ml, a gas (air) was introduced into the reaction system, a pH of the reaction system was adjusted to be 8.0, the reaction system was controlled to be subjected to a reaction at a temperature of 40° C., and the reaction was finished after 15 hours of the reaction. The resulting reaction solution was detected, and the results showed that the concentration of (2S,3R)-p-methylsulfonylphenylserine was 78.649 g/l, the final volume was 104 ml, and a conversion rate of (2S,3R)-p-methylsulfonylphenylserine was 96.85%.

### Example 8

The reaction solutions obtained in Examples 3-7 were mixed, a pH was adjusted to be 3.0, centrifugation was performed to remove proteins and impurities, followed by decolorization by addition of activated carbon, and filtration, a pH of a filtrate collected after the filtration was adjusted to be 7.0 and the filtrate was concentrated by rotary evaporation, magnesium sulfate with a molar mass equal to that of the product was added after the concentration, stirring was performed to dissolve magnesium sulfate, a pH was adjusted to be about 9.5, the temperature was reduced for low temperature precipitation, and suction filtration was performed to obtain a magnesium salt form of (2S,3R)-p-methylsulfonylphenylserine with a de value of 98%.

### Example 9

Aldehyde oxidase exists not only in microorganisms but also in plants. To explore whether aldehyde oxidase contained in the plants can play the role of eliminating acetaldehyde, the present disclosure also studied some plants, and milled plant juice was used instead of aldehyde oxidase. The specific operation in this example was as follows:

Extraction of plant branches and leaves: 25 g of stems and leaves of azalea plants were harvested and washed, 100 ml of ddH2O was added to crush the stems and leaves of the azalea plants by milling, a milled solution was filtered with a gauze to remove plant residues, then the filtered solution was centrifuged at 10000 rpm for 10 min, and a supernatant obtained after the centrifugation was used as a reaction solvent instead of acetaldehyde oxidase for a reaction.

Reaction system: 3 g of p-methylsulfonylbenzaldehyde, 2.25 g of L-threonine, 0.1 g of pyridoxal phosphate, 10 ml of transaldolase (purchased from Taizhou Lingfeng Biotechnology Co., Ltd., LF145), 5 ml of catalase, and 85 ml of the azalea branch and leaf extract were added into a clean reaction flask, with a total volume of 100 ml, a gas (air) was introduced into the reaction system, a pH of the reaction system was adjusted to be 7.0, a reaction was carried out for 11 h under the condition that the temperature of the reaction system was controlled to be 35° C., after the reaction was finished, the resulting reaction solution was detected, and the results showed that the concentration of (2S,3R)-p-methylsulfonylphenylserine was 41.029 g/l, the final volume was 94 ml, and a conversion rate of (2S,3R)-p-methylsulfonylphenylserine was 91.33%.

### Example 10

In order to better illustrate that the addition of the plant extract in Example 9 also has a good promotion effect on the reaction, a corresponding control experiment was carried out in this example. As a control, a control group adopted transaldolase alone for a reaction.

Reaction system: 3 g of p-methylsulfonylbenzaldehyde, 2.25 g of L-threonine, 0.1 g of pyridoxal phosphate, 10 ml of transaldolase (purchased from Taizhou Lingfeng Biotechnology Co., Ltd., LF145), and 90 ml of ddH2O were added into a clean reaction flask, with a total volume of 100 ml, a gas (air) was introduced into the reaction system, a pH of the reaction system was adjusted to be 7.0, and after a reaction was carried out for 11 h under the condition that the temperature of the reaction system was controlled to be 35° C., the reaction was finished. The resulting reaction solution was detected, and the results showed that the concentration of (2S,3R)-p-methylsulfonylphenylserine was 24.442 g/l, the final volume was 107 ml, and a conversion rate of (2S,3R)-p-methylsulfonylphenylserine was 61.93%.

### Example 11

Extraction of plant branches and leaves: 25 g of stems and leaves of Schefflera octophylla plants were harvested and washed, 100 ml of ddH2O was added to crush the stems and leaves of the Schefflera octophylla plants by milling, a milled solution was filtered with a gauze to remove plant residues, then the filtered solution was centrifuged at 10000 rpm for 10 min, and a supernatant obtained after the centrifugation was used as a reaction solvent instead of acetaldehyde oxidase for a reaction.

Reaction system: 3 g of p-methylsulfonylbenzaldehyde, 2.25 g of L-threonine, 0.1 g of pyridoxal phosphate, 10 ml of transaldolase (purchased from Taizhou Lingfeng Biotechnology Co., Ltd., LF145), 5 ml of catalase (purchased from Novozymes and having an enzyme activity of 60000 SCIU/mL), and 85 ml of the Schefflera octophylla branch and leaf extract were added into a clean reaction flask, with a total volume of 100 ml, a gas (air) was introduced into the reaction system, a pH of the reaction system was adjusted to be 7.0, and after a reaction was carried out for 11 h under the condition that the temperature of the reaction system was controlled to be 35° C., the reaction was finished. The resulting reaction solution was detected, and the results showed that the concentration of (2S,3R)-p-methylsulfonylphenylserine was 22.266 g/l, the final volume was 108 ml, and a conversion rate of (2S,3R)-p-methylsulfonylphenylserine was 56.95%.

### Example 12

In order to better illustrate that the addition of the plant extract in Example 11 also has a good promotion effect on the reaction, a corresponding control experiment was carried out in this example. As a control, a control group adopted transaldolase alone for a reaction.

Reaction system: 3 g of p-methylsulfonylbenzaldehyde, 2.25 g of L-threonine, 0.1 g of pyridoxal phosphate, 10 ml of transaldolase (purchased from Taizhou Lingfeng Biotechnology Co., Ltd., LF145), and 90 ml of ddH2O were added into a clean reaction flask, with a total volume of 100 ml, a gas (air) was introduced into the reaction system, a pH of the reaction system was adjusted to be 7.0, and after a reaction was carried out for 11 h under the condition that the reaction temperature was controlled to be 35° C., the reaction was finished. The resulting reaction solution was detected, and the results showed that the concentration of (2S,3R)-p-methylsulfonylphenylserine was 19.266 g/l, the final volume was 104 ml, and a conversion rate of (2S,3R)-p-methylsulfonylphenylserine was 47.45%.

### Example 13

6 g of p-methylsulfonylbenzaldehyde, 5.0 g of L-threonine, 0.3 g of pyridoxal phosphate, 15 ml of transaldolase (purchased from Taizhou Lingfeng Biotechnology Co., Ltd., LF145), 20 ml of aldehyde oxidase (a supernatant obtained after wall breaking and centrifugation, i.e., the enzyme solution obtained in Example 1), 65 ml of ddH2O and 5 ml of catalase (purchased from Novozymes and having an enzyme activity of 60000 SCIU/mL) were added into a clean reaction flask, with a total volume of 100 ml, a pH of the system was adjusted to be 8.0, and after a reaction was carried out at a temperature controlled to be 38° C. for 18 h while introducing a gas (air), the reaction was finished. The resulting reaction solution was detected, and the results showed that the concentration of (2S,3R)-p-methylsulfonylphenylserine was 77.312 g/l, the final volume was 94 ml, and a conversion rate of (2S,3R)-p-methylsulfonylphenylserine was 86.05%.

### Example 14

6 g of p-methylsulfonylbenzaldehyde, 4.0 g of L-threonine, 0.2 g of pyridoxal phosphate, 15 ml of transaldolase (purchased from Taizhou Lingfeng Biotechnology Co., Ltd., LF145), 20 ml of aldehyde oxidase (a supernatant obtained after wall breaking and centrifugation, i.e., the enzyme solution obtained by the method of Example 1), 65 ml of ddH2O and 5 ml of catalase (purchased from Novozymes and having an enzyme activity of 60000 SCIU/mL) were added into a clean reaction flask, with a total volume of 100 ml. A gas (air) was introduced into the reaction system, a pH of the reaction system was adjusted to be 7.5, the reaction system was controlled to be subjected to a reaction at a temperature of 35° C., and the reaction was terminated after 20 hours of the reaction. The resulting reaction solution was detected, and the results showed that the concentration of (2S,3R)-p-methylsulfonylphenylserine was 77.393 g/l, the final volume was 97 ml, and a conversion rate of (2S,3R)-p-methylsulfonylphenylserine was 89.89%.

### Example 15

6 g of p-methylsulfonylbenzaldehyde, 4.5 g of L-threonine, 0.1 g of pyridoxal phosphate, 22 mg of transaldolase lyophilized powder (the activity of transaldolase being 18.6 U/mg), 15 ml of acetaldehyde oxidase (a supernatant obtained after wall breaking and centrifugation, i.e., the enzyme solution obtained by the method of Example 1), 65 ml of ddH2O and 5 ml of catalase (purchased from Novozymes and having an enzyme activity of 60000 SCIU/mL) were added into a clean reaction flask, a gas (air) was introduced into the reaction system, a pH of the reaction system was adjusted to be 7.0, and after the reaction system was controlled to be subjected to a reaction at a temperature of 35° C. for 23 hours, the reaction was terminated. The resulting reaction solution was detected, and the results showed that the concentration of (2S,3R)-p-methylsulfonylphenylserine was 89.421 g/l, the final volume was 76 ml, and a conversion rate of (2S,3R)-p-methylsulfonylphenylserine was 80.47%.

The specific examples described in the present disclosure are merely illustrative of the spirit of the present disclosure. Those skilled in the art to which the present disclosure belongs may make various modifications or supplements or similar substitutions to the specific examples described without departing from the spirit of the present disclosure or being beyond the scope defined by the appended claims.

Although the present disclosure has been described in detail and specific examples have been cited, it will be apparent to those skilled in the art that various changes and modifications may be made without departing from the spirit and scope of the present disclosure.

What is claimed is:

1. A method for preparing (2S,3R)-p-methylsulfonylphenylserine, comprising a step of:
carrying out a reaction between p-methylsulfonylbenzaldehyde, L-threonine, and pyridoxal phosphate in an oxygen-containing environment under a combined action of a transaldolase and an acetaldehyde oxidase to obtain the (2S,3R)-p-methylsulfonylphenylserine.

2. The method for preparing the (2S,3R)-p-methylsulfonylphenylserine according to claim 1, wherein the acetaldehyde oxidase is selected from one or more of an acetaldehyde oxidase having the amino acid sequence shown in SEQ ID NO: 1, an azalea plant extract, and a Schefflera octophylla plant extract.

3. The method for preparing the (2S,3R)-p-methylsulfonylphenylserine according to claim 1, wherein the oxygen-containing environment is an environment introduced with air or oxygen.

4. The method for preparing the (2S,3R)-p-methylsulfonylphenylserine according to claim 1, wherein a catalase is also added into raw materials of the reaction.

5. The method for preparing the (2S,3R)-p-methylsulfonylphenylserine according to claim 1, wherein the reaction is carried out at a temperature of 30° C.-40° C.

6. The method for preparing the (2S,3R)-p-methylsulfonylphenylserine according to claim 1, wherein the reaction is carried out in water.

7. The method for preparing the (2S,3R)-p-methylsulfonylphenylserine according to claim 1, wherein the reaction is carried out at a pH of 6.5-9.0.

8. The method for preparing the (2S,3R)-p-methylsulfonylphenylserine according to claim 1, wherein a mass ratio of the p-methylsulfonylbenzaldehyde to the L-threonine to the pyridoxal phosphate is 6.0:(4.0-5.0):(0.1-0.3).
